(19) United States
(12) Patent Application Publication
(10) Pub. No.: US 2016/0198960 A1
(43) Pub. Date: Jul. 14, 2016

(54) PET ANIMAL COLLAR FOR HEALTH & VITAL SIGNS MONITORING, ALERT AND DIAGNOSIS

(71) Applicant: Avi MENKES, Ramat Hasharon (IL)

(72) Inventor: Avi MENKES, Ramat Hasharon (IL)

(73) Assignee: PatPace Ltd., Ramat-Hasharon (IL)

(21) Appl. No.: 15/077,968

(22) Filed: Mar. 23, 2016

Related U.S. Application Data
(63) Continuation of application No. 13/400,595, filed on Feb. 21, 2012.
(60) Provisional application No. 61/507,679, filed on Jul. 14, 2011.
(60) Provisional application No. 61/522,327, filed on Aug. 11, 2011.

(51) Int. Cl. A61B 5/0205; A61B 7/04; A61B 5/00; A01K 29/00; A01K 27/00

(52) U.S. Cl. CPC A61B 5/02055; A01K 29/005; A01K 27/001; A01K 27/009; A61B 5/6822; A61B 5/6831; A61B 5/72; A61B 5/7465; A61B 7/04; A61B 5/7405; A61B 2503/40; A61B 2562/0219; A61B 2560/0252; A61B 2562/0223; A61B 2560/0475

(57) ABSTRACT

A collar for pet animals may have sensor elements remotely actuatable to measure vital signs of the animal (such as respiration, pulse, temperature and movement) and a processor that can interpret the results of multiple vital sign readings. A two way communication device alerts the pet owner, veterinarian or authorities. A veterinarian can remotely take a particular vital sign measurement when alerted. The sensor elements embedded in the collar's band has at least one elastic pin extending toward the animal's neck to gather data processed on the collar or remotely. To improve STN ratio, an elastic layer may absorb noise from friction due to movement of the animal's head. The collar may adjust the tightness of the band for taking vital sign readings. For example pump may injects air through a tubular compertment running along the circumference of the band. A safety mechanism may release the collar.

This application claims the priority of U.S. Provisional Application No. 61/507,679 filed Jul. 14, 2011 and US Provisional Application No. 61/522,327 filed Aug. 11, 2011

### FIELD AND BACKGROUND OF THE INVENTION

The present invention relates to apparatuses and methods for monitoring vital signs and health of animals, and, more particularly for monitoring the health and vital signs of pet animals, such as dogs and cats, and doing so using a specially designed collar.

When animals, including pets such as dogs and cats, are sick they tend by nature to withdraw and hide since they feel defenseless. This behavior makes treatment of the animal significantly more difficult. With regard to pet animals, such as dogs and cats, it is known for veterinarians to check the vital signs of a sick dog or a sick cat. However, this tends to occur long after the animal has contracted the medical problem either because the dog or cat was hiding and/or because it takes time to reach the veterinarian. Early detection is often not achieved yet is very important in order to achieve less suffering of the pet and less likelihood of acute disease, which can develop if detection occurs late. Regarding ear infections in a dog, for example, according to Veterinary Pet Insurance (VPI), this is the most common medical condition affecting dogs in 2010 and “identifying changes or redness early will help dogs and cats avoid more irritating, painful and expensive ear infections. The longer a problem is allowed to persist, the more difficult it is to treat.”.

Moreover, stray dogs and cats, as well as dogs and cats whose owners are not constantly with them as a practical matter, and dogs and cats whose owners are on vacation, are more vulnerable to contracting an illness, exhibiting hiding behavior patterns and decreasing the chances of timely medical intervention.

In addition, monitoring the health of captive animals, for example animals in zoos, is an arduous and expensive task.

There is a compelling need to have an apparatus and method that will provide early detection and diagnosis of pet animals such as dogs and cats.

### SUMMARY OF THE PRESENT INVENTION

One aspect of the present invention is a collar for monitoring vital signs of a pet animal, comprising a band having a layer of an elastic material, the band for positioning on a neck of the animal; at least one sensor element at different points of the band, each sensor element having at least one elastic pin projecting from the band towards the neck, the at least one element configured to measure at least one bioparameter from the following bioparameters: temperature, heart rate, respiration rate, movement, the band having a first position for use in measuring the at least one bioparameter and a second position for use when not measuring the at least one bioparameter, the second position tighter around the neck than the first position

A further aspect of the present invention is a collar for monitoring vital signs of a pet animal, comprising a tubular band having a layer of an elastic material, the band for positioning on a neck of the pet animal; at least three sensor elements at different points of the band, each sensor element having at least one elastic pin projecting from the band towards the neck, each elastic pin for penetrating fur of the pet animal without causing the pet animal discomfort, the at least three sensor elements for measuring at least two bioparameters from temperature, heart rate, respiration rate, movement, each of the at least three sensor elements configurable remotely, the adjustable length band having a first position for measuring a first bioparameter and a second position for monitoring a second bioparameter;

an actuator and a pump for pumping air into the tubular band at different amounts to tighten and loosen the band between a plurality of tightness positions including the first and second positions, a processor affixed to the collar and hard-wired to each of the at least three sensor elements and the motor, the processor for receiving sensor data from the sensor elements and for communicating data to a telecommunications system and the processor for controlling the motor

A still further aspect of the present invention is a method of monitoring vital signs of a pet animal, comprising providing a collar having a band whose tightness is configured to be adjusted remotely; implanting into the collar sensor elements at different points along a circumference of the band, each implanted sensor element having at least one elastic pin projecting from the band towards the neck, the sensor elements for measuring at least heart rate and respiration rate; configuring different tightness positions of the band, a first tightness position for when a vital sign is measured and a second tightness position for when vital signs are not being measured; and either (i) configuring a processor on the collar that is in electronic communication with the sensor elements to determine vital signs of the pet animal and transmitting a signal from the collar to a remote station, the signal reflecting vital sign measurements or (ii) configuring a remote processor that is in electronic communication with the sensor elements to determine vital signs of the pet animal, the signal reflecting vital sign measurements.

A yet still further aspect of the present invention is a method of monitoring vital signs of a pet animal, comprising providing a collar having a band whose tightness is configured to be adjusted remotely; implanting, into the collar an array of sensor elements at different points along a circumference of the band, each implanted sensor element having at least one elastic pin projecting from the band towards the neck, the sensor elements for measuring vital signs of the pet animal including at least respiration rate and heart rate; configuring a tightness of the band sufficient for measuring different vital signs by different sensor elements without the band being too tight that the pet animal is discomforted; and either (i) configuring a processor on the collar that is in electronic communication with the sensor elements to determine vital signs of the pet animal and transmitting a signal from the collar to a remote station, the signal reflecting vital sign measurements or (ii) configuring a remote processor that is in electronic communication with the sensor elements to determine vital signs of the pet animal, the signal to the processor reflecting vital sign measurements

A further aspect of the present invention is a pet animal collar for monitoring vital signs of a pet animal, comprising an adjustable length band having a layer of an elastic material, the band for positioning on a neck of the pet animal; at least four sensor elements at different points of the band, each sensor element having at least one elastic pin projecting from the band towards the neck and having a power source, the at least one sensor elements for measuring at least two bioparameters from temperature, heart rate, respiration rate, blood pressure, movement, each of the at least four sensor elements configurable remotely; and a processor affixed to the collar and in electronic communication with each of the at least four sensor elements for controlling a timing of an “ON” status of each sensor sufficient to trigger taking of a vital sign measurement, the processor configured to calculate the timing based on power requirements of the at least four sensors and a lifespan of the power source, the processor for receiving sensor data from the sensor elements and for communicating vital sign data to a remote location

These and other features, aspects and advantages of the present invention will become better understood with reference to the following drawings, descriptions and claims.

### BRIEF DESCRIPTION OF THE DRAWINGS

Various embodiments are herein described, by way of example only, with reference to the accompanying drawings, wherein:

FIG. 1 is a schematic diagram of a sensor element having multiple elastic pins, in accordance with one embodiment of the present invention;

FIG. 2a is a schematic side view of a collar around a pet's neck and including pins of sensor elements projecting toward the neck and showing a controller, in accordance with one embodiment of the present invention;

FIG. 2b is a schematic side view of a collar showing pins of sensor elements projecting in a direction of a neck (not shown) of the pet, in accordance with one embodiment of the present invention;

FIG. 3 is a high level scheme of a sensor array and associated electronics, the electronics inside a controller, in accordance with one embodiment of the present invention;

FIG. 4 is a schematic of the architecture of an overall system, in accordance with one embodiment of the present invention;

FIG. 5 is a flow chart showing a method, in accordance with one embodiment of the present invention;

FIG. 6 is a flow chart showing a further method, in accordance with one embodiment of the present invention; and

FIG. 7 is a diagram showing a mechanism for adjusting a tightness of a collar, in accordance with one embodiment of the present invention.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

The present invention generally provides a collar for pet animals such as dogs and cats. The collar may have sensor elements that can be activated remotely to check vital signs of the animal (such as respiration, pulse, temperature and movement) and a processor that can interpret the results of multiple vital sign readings. The collar may also have a two way communication device attached or integrated thereto that can alert the pet owner, a veterinarian or the authorities, when appropriate, that a pet animal is suffering from a particular condition or is exhibiting suspicious behavior or movements. This way, a veterinarian can remotely take a particular vital sign measurement when alerted of the data by signalling the processor to actuate a particular sensor element. The sensor elements embedded in the band of the collar gather data that can be processed on the collar itself or transmitted to a remote terminal, which can be a home computer, a hand-held device, or a main server computer. In order to dramatically improve signal to noise ratio (STN), an elastic layer may absorb noise from friction due to movement of the animal's head. The collar also may have the ability to adjust the tightness of the band around the neck of the pet animal to make the collar in condition to take a vital sign reading, or to make it suitable for a particular vital sign measurement. This may be accomplished, for example through use of a pump that injects air through a tubular compertment running along the circumference of the band of the collar, A safety mechanism releases the collar.

In contrast to prior art pet animal collars, which do not measure vital signs, the pet collar of the present invention may measure vital signs of the pet animal. For example, it may measure, heart rate, respiration rate, blood pressure, temperature, movement, etc. In further contrast to the prior art pet animal collars, which are not automatically or remotely adjustable, the animal pet collar of the present invention may be automatically and remotely adjustable in tightness around the pet's neck. This helps the collar measure different vital sign parameters depending on how tight or loose the collar is. For example, the collar may be tightened when the blood pressure is measured and loosened when respiration rate is measured. In still further contrast to prior art pet collars, the collar may include a processor and may interpret the interdependence of the vital sign measurements made by the sensor array to arrive at a tentative diagnosis that may be relayed to a veterinarian, the pet owner and/or to the authorities. In still further contrast to the prior art, the collar may have two-way communication so that a veterinarian can instruct the collar to measure a particular vital sign remotely. In contrast to prior art dog or pet collars, which may be adjustable in tightness, the collar of the present invention may be adjustable in tightness remotely by pumping air (or conversely by withdrawing or not pumping air) into an area along a length of the collar's band. In still further contrast to the prior art animal pet collars, such as dog collars, in which signal to noise ratio precludes remote telecommunication reception of vital sign parameters, the collar of the present invention may include a layer of elastic that improves the signal to noise ratio by absorbing friction from constant movement of the dog or pet's head. In contrast to the prior art collars, the collar of the present invention may also have a GPS and communications system for alerting remote personnel so that if the pet animal is ill, or if a captive animal in a zoo escapes its enclosure, an immediate alarm can be sounded and an alert transmitted to deisgnated authorities and veterinarians.

The principles and operation of a method and apparatus for a pet animal collar for health & vital signs monitoring, alert and diagnosis may be better understood with reference to the drawings and the accompanying description.

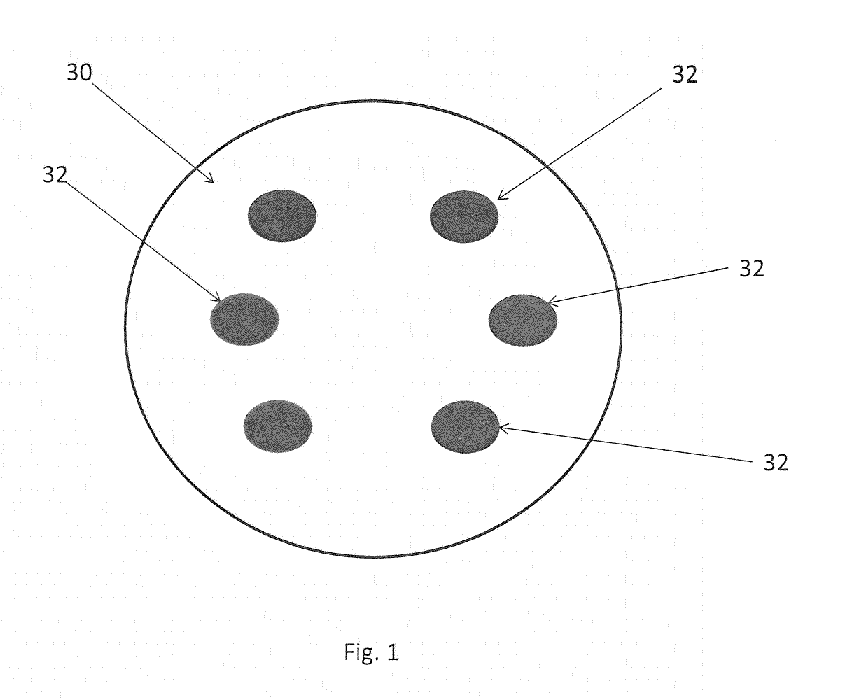

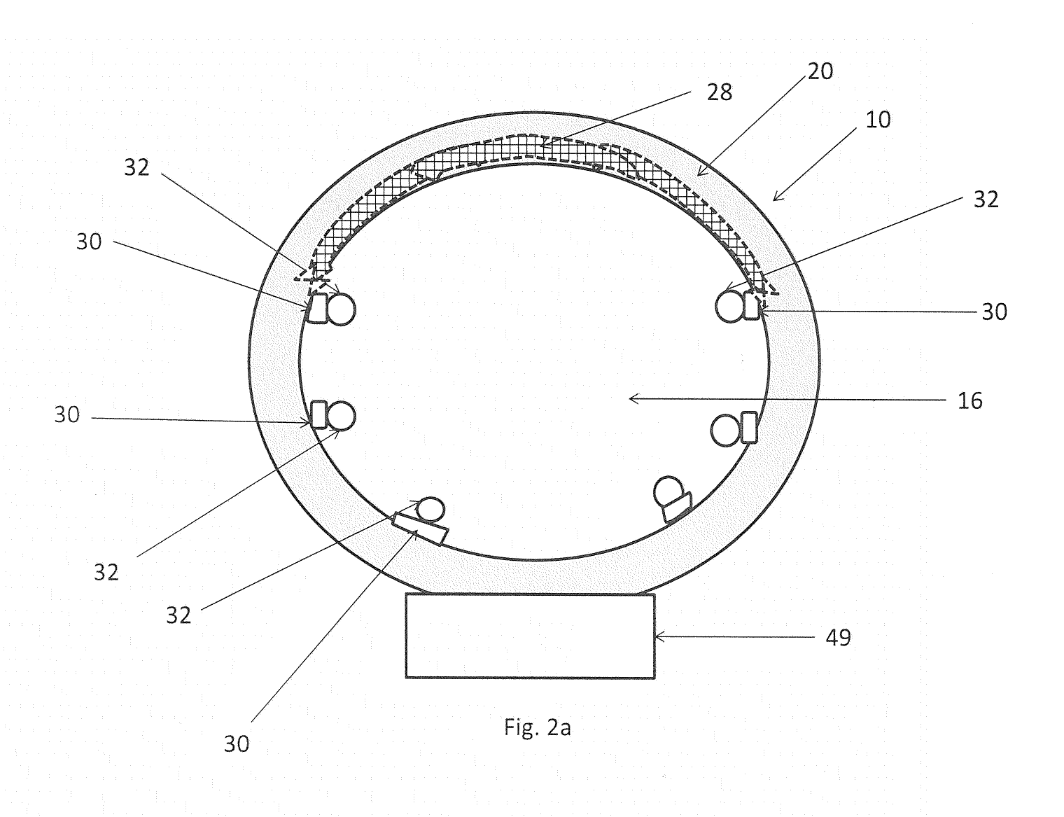

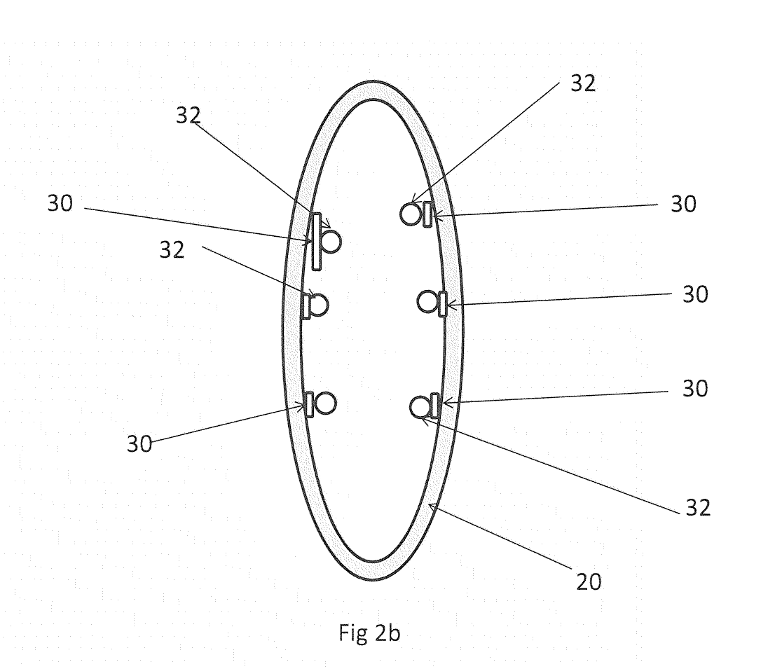

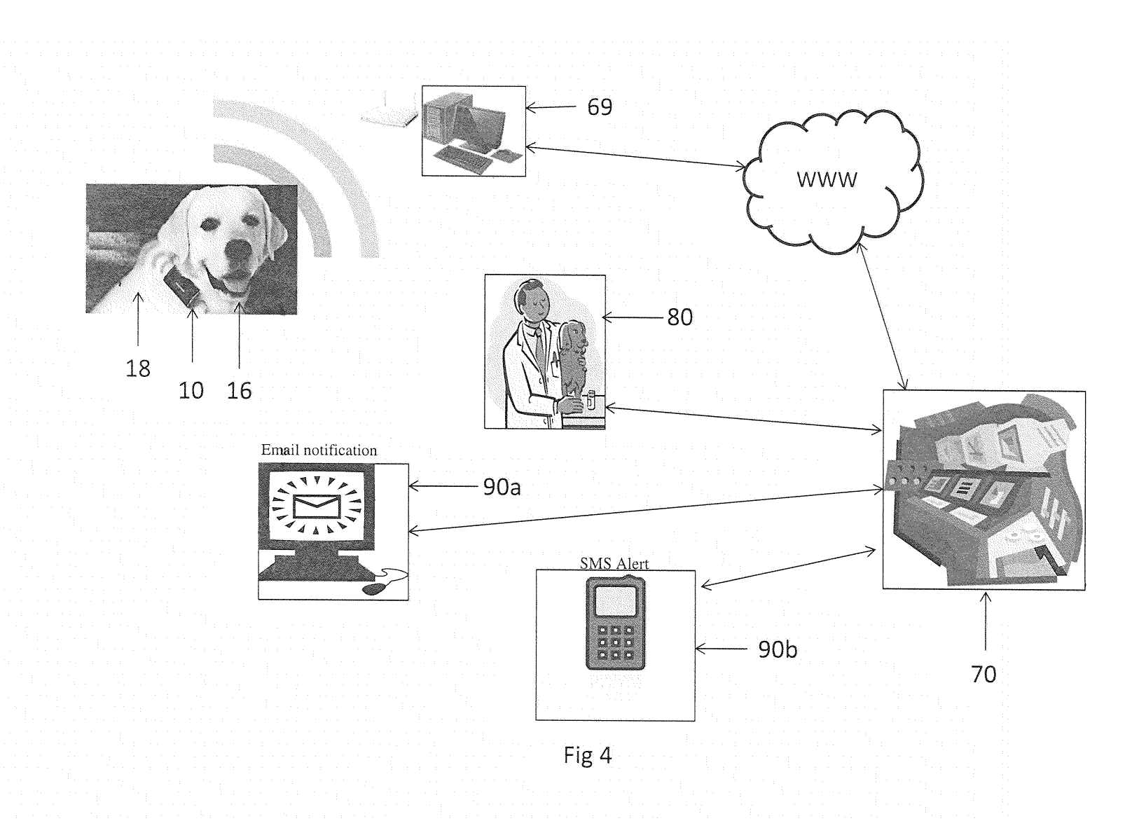

As seen from FIGS. 1, 2a and 2b, a collar 10 for monitoring vital signs of a pet animal (18 in FIG. 4) may include a band 20. Band 20 may have a layer of an elastic material 28. A portion of an elastic layer 28 that may extend around the entire band 20 (or portions of the band 20) is shown in FIG. 2a. The elastic material 28 may be for cushioning repetitive instances of friction against the collar 10 from the head of the pet animal. Band 20 may be for positioning on or adjacent the neck of the pet animal. Band 20 (and collar 10) may be approximately two inches wide and may cover an entire circumference of the neck of the pet (or alternatively most or a portion of this circumference). There may be sensors 30, for example four or more sensor elements 30 at different points of the band, preferably at different points along a length or circumference of band 20. There may be other numbers of sensor elements, such as one, two, three, five, six, seven, eight, nine or ten and more.

As shown in FIG. 1, and in FIG. 2, each sensor element 30 may have at least one elastic pin 32 projecting from the band 20 towards the neck 16 of the pet animal 18. The pins 32 may be made of silicone and may touch the skin of the pet and absorb the noise from friction while conducting the signal. Each elastic pin 32 may penetrate the fur on the neck of the animal without causing the animal discomfort. This may be arranged by configuring the length of the pin 32 (its length from the sensor element 30 substantially perpendicularly toward the neck of the pet animal) and thereby controlling how far the pin projects toward the direction of the neck of the pet animal. The comfort of the pet animal may be verified by testing the collar on various pet animals of the particular species. This may also be arranged by adjusting the tightness of band 20 around the neck of the pet, as discussed below.

In general, sensor elements 30 may be at least one sensor element 30 designed or configured to measure at least one bioparameter from among temperature, heart rate, respiration rate and movement. Alternatively, the sensor element may be for measured a different vital sign. There could be more sensor elements and more bioparameters. For example, the at least one sensor element 30 may comprise at least two sensor elements 30 that may be configured or designed to measure at least two bioparameters from among temperature, heart rate, respiration and movement. Alternatively, the at least two sensor elements 30 may be for measuring at least two bioparameters from among temperature, heart rate, respiration rate and movement (or alternatively other vital signs). One sensor element may measure multiple bioparameters, for example, in the case of an acoustic sensor that measures respiration rate and heart rate. The at least two sensor elements may comprise four or more sensor elements designed to measure four or more bioparameters or specifically those four: temperature, heart rate, respiration rate and movement. In some preferred embodiments, the array of sensor elements 30 are designed to measure one or two bioparameters (in other preferred embodiments three or four) from the following bioparameters: temperature, heart rate, respiration rate, movement (for example horizontal and vertical movement).

The sensor elements 30 may be designed or configured to measure at least two different vital sign bioparameters as well as to measure certain bioparameters, such as movement, that may be useful in understanding a pet's vital signs when combined with other vital sign bioparameters. Each of the various sensor elements 30 on the band 20 may be designed for measuring a different vital sign parameter or in some cases there may be more than one sensor element measuring a particular vital sign bioparameter or more than one vital sign measured by a particular sensor element 30.

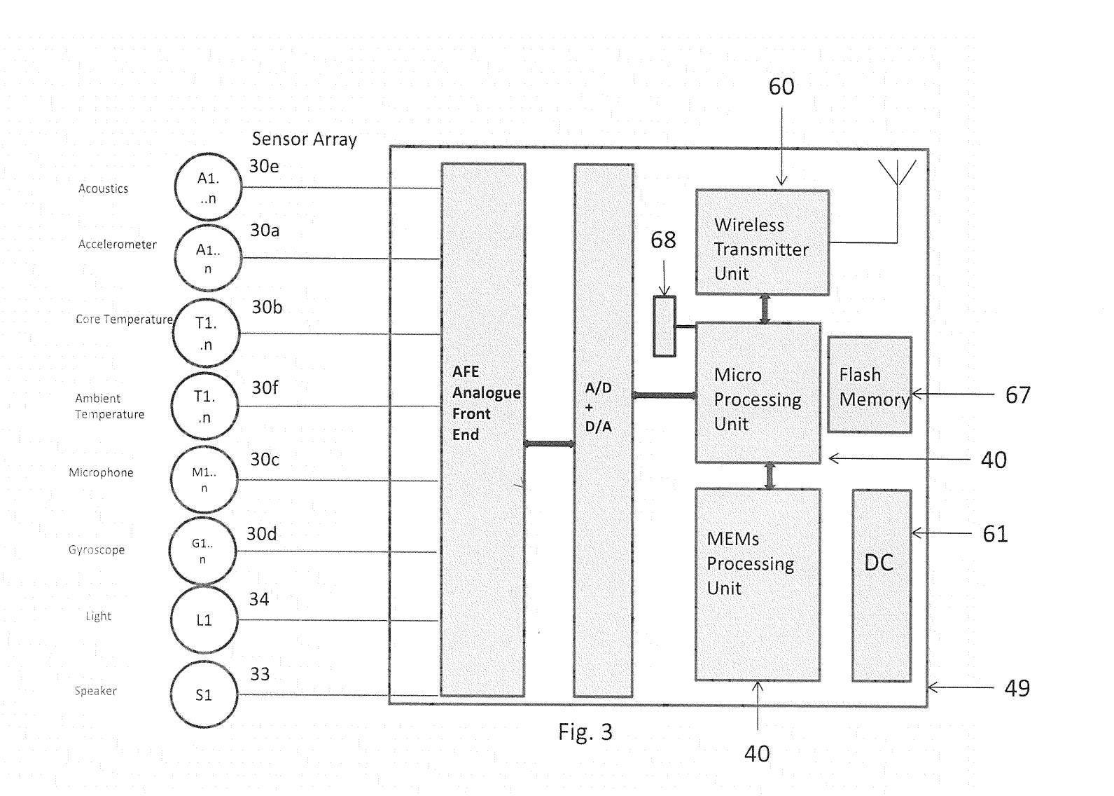

As shown in FIG. 3, sensor array 30 may include an acoustic sensor element 30e for measuring pulse (heart rate) and an acoustic sensor for measuring respiration rate. As further shown in FIG. 3, sensor array 30 may include an accelerometer 30a to measure movement and vibrations of air traveling through the pet's air canals during inhaling and exhaling motions as well as the movement of blood traveling through the main blood vessels across the pet's neck. Sensor array 30 may also include a temperature sensor 30b to measure the temperature of the pet's body and an ambient temperature sensor 30f to measure the ambient temperature.

Sensor array 30 may also include a microphone 30c. Sensor array 30 may further include a microphone 30c to listen to special noises made by a pet animal, for example a dog. In the case of a dog, there are about twenty-six separate sounds that they normally make. These include the following: barking sounds (including guarding/warning bark, alarm barking, playing, anxiety, need bark), yelping, growling, howling, eating, drinking, breathing (including normal breathing through the nose (inspiration and expiration), open-mouthed breathing, dry cough, wet cough, stertor, stridor, laryngeal paralysis, wheezing, rales/crackles, bronchio-vesicular sounds), vomiting/retching, regurgitation, grunting, groaning, and panting. Furthermore, each of these types of sounds may be further subdivided into sounds of those type made by a small dog, made by a large dog, made by a deep-chested dog and made by a puppy dog. Accordingly, the sounds picked up by microphone 30c may be interpreted by a processor 40 having an associated memory storage 67 (FIG. 3) of collar 10 or a remote processor of a remote computer termional 69 (FIG. 4) and/or by a processor having access to a dedicated or remote database to determine the type of sound and its interdependence with other vital sign bioparameters in order to arrive at a tentative diagnosis, to determine whether an alert is justified or to suggest treatment.

The sensor array 30 may also include a gyroscope 30d for capturing the vertical and/or horizontal movement of the pet. In the case of dogs, there are numerous basic dog postures that provide information as to what the dog is doing and thereby assist in interpreting vital sign measurements to arrive at a tentative diagnosis. The following basic dog postures that may be detected by sensor elements 30, for example a gyroscope, an accelerometer and/or a magnetometer: lying down laterally, lying down sternally (head up/down), lying on back, sitting, standing on four legs, standing on back legs, jumping, trotting, running, eating/drinking, urinating (male/female), defecating, limping hind leg, limping front leg, scratching hind leg, shaking leg, turning to lick, and stretching. The processor 40 make receive this information from the sensors 30 and utilize it in reaching a conclusion that it transmits remotely to the appropriate destination.

Each of the sensors 30 may be activated, de-activated, fine-tuned, set for predetermined repeated intervals or otherwise calibrated or controlled remotely, and in some embodiments also manually by a person located at the collar 10. “Remotely” means remote from the collar 10 and may include by a person in a vital sign monitoring station or a remotely stationed veterinarian or a medical center or the pet owner or the authorities or any other suitable location.

As shown in FIG. 3, collar 10 may further include a remotely-actuatable speaker 33 for communicating sounds to the pet animal remotely and may include a remotely actuatable light 34 (such as an LED or other light source) for illuminating the pet animal to those seeking to locate it. The speaker 33 and light 34 may also be actuatable manually in person. The speaker 33 and light 34 may be situated on or attached to the band 20 and may be included in the array shown in FIG. 2 (even though the light is not a sensor).

The adjustable length band 20 may have a first position (or a first tightness position) for use when the collar is worn and no vital sign bioparameters (or any bioparameters) are being measured and a second position (or a second tightness position) for use when the collar is worn and one or more vital sign bioparameters are being measured. For example, the second position may indicate that the band 20 is tighter around the neck than the first position. For example, as seen in FIG. 2a, the elastic pins 32 may penetrate the fur of the animal's neck when the band 20 is tighter and the pins 32 may make sufficient contact with the neck 16 of the animal 18 to be able to measure and record vital signs, such as respiration rate, heart rate, pulse, temperature or other vital signs. The number of tightness positions may exceed two and may be other discrete integers that are equal to (or even greater than) the number of different sensor elements for measuring different bioparameters of the pet.

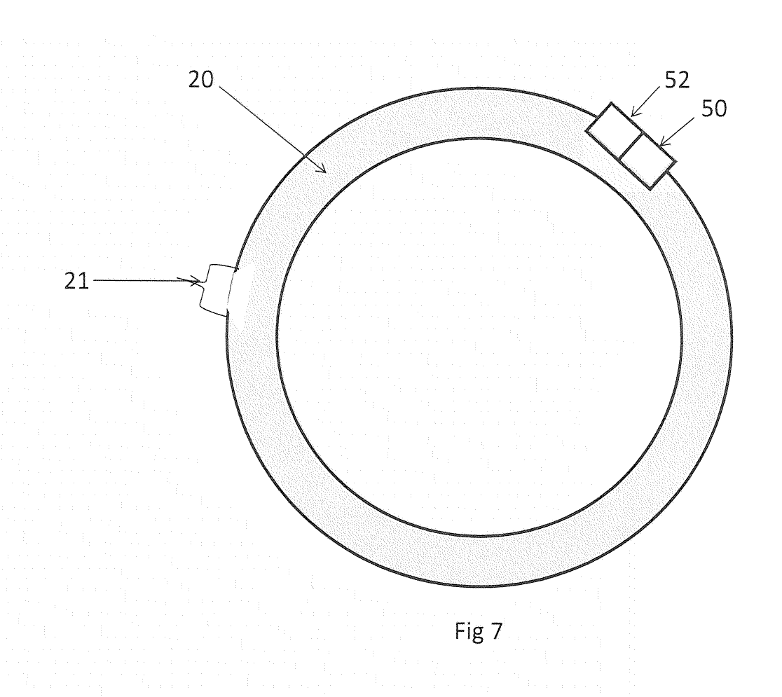

As seen schematically from FIG. 7, collar 10 may also include a motor 50 and a pump 52 for pumping air at different amounts into band 20 for example all along a length of the band 20 that is normally flattened unless air is pumped into it. For this purpose, band 20 may be configured to be tubular with an internal space for air. As a result, pump 52 and motor 50 controlled by controller 49 including processor 40 may inject or withdraw air or another fluid into band 20 in order to tighten and loosen the band 20, and hence to tighten or loosen collar 10, around the pet's neck. There may be several tightness positions. The tightness positions may include a first position that is tighter and therefore more appropriate for taking vital sign bioparameter measurements which require the pins of sensor elements to be in contact with the skin of the neck of the pet. The tightness positions may also include a second position appropriate for when the vital sign measurements are not being taken. In that case, the collar 10 can be looser. In some embodiments, the tightness positions can include a third position, where the band is at its tightest, for measuring particular vital signs that require such tightness. In some embodiments, this may include blood pressure measurements.

As seen schematically from FIG. 7, band 20 may have a release mechanism 21 that activates if the pet is in danger, for example as a result of the collar 10 being too tight. The release mechanism can be triggered based on reaching a threshold level of a vital sign or a physiological data such as a movement that alone or in combination indicates danger to the dog's breathing or other danger based on an algorithm. In one preferred embodiment, the release mechanism is a latch 21 or other attachment element connecting two parts of the length of the band 20 to one another. The release mechanism may in some embodiments be an aperture that is uncovered to release air from the internal space of band 20 in the event of danger. The release of air loosens collar 10. The attachment element may be remotely actuated, for example if the attachment element comprises a small latch with a magnetic closure means that is remotely actuatable as “ON” of “OFF’ by the processor 40 in the controller 49 on the collar 10 or remotely.

As seen from FIG. 2A, collar 10 may also include a controller 49 that includes a processor 40 that may be affixed to the collar 10 for example in a housing (not shown) attached to the collar 10. As shown in FIG. 3, processor 40 may also include a processing unit having MicroElectro Mechanical Systems (“MEMS”) technology. As also shown from FIG. 3, processor 40 may be hard-wired or otherwise in electronic communication with each of the sensor elements 30 and the motor 52 (if the collar 10 includes a pump 50). Processor 40 may be configured to receive a signal representing data sensed by one or more of the sensor elements 30 and may be configured to analyze the data and communicate vital sign determinations and other data to a telecommunications system. Processor 40 may also control the motor 52 for adjusting the tightness of the band 20 in the event the collar has a pump 50 and motor 52. The vital sign data measured by the sensor elements 30 of collar 10 may be relayed to and interpreted by a processor 40. Processor 40 may execute algorithms to interpret a collection of the physiological data sensed by the sensor elements and the interdependence of the vital sign data from the sensor elements and arrive at a tentative diagnosis. The vital sign data may also include physiological data such as data about the movement of the pet animal (or other physiological data such as the saltiness of the animal's skin) since this physiological data, when combined with fundamental vital signs such as breathing rate, respiration rate, pulse, temperature, etc. may be useful in diagnosis by the veterinarian or remote computer server for the automatic temperoray diagnosis by the processor 40.

Controller 49 may also include a memory storage for storing health information history of the pet animal, the memory storage accessible by the processor 40. The memory storage can be a flash memory 67 as shown in FIG. 3 or other memory storage devices known in the art.

As shown in FIG. 4, collar 10 may include a communication device 60 such as a wireless transmitter unit, that may be accompanied by a receiving unit 66 forming a two-way communication device for communication to a remote station 70 (FIG. 4) which may include a computer server pre-programmed to interact with the processor 40 or the remote station 70 may communication with or include a veterinarian 80 (FIG. 4) who can remotely measure vital signs using the collar's processor to select particular sensor elements to be activated to measure vital signs of the pet. The remote station 70 may also alert a pet owner or the authorities by sending an email communication 90a (FIG. 4) or an SMS alert 90b (FIG. 4). The communication device 60 may also incorporate short range or long range wireless communication technology such as UHF, Wi-Fi, Bluetooth, etc. and cellular technology.

The collar 10 and/or server computer 70 or other part of the system may issue an alert based on predefined parameters (e.g. unique prior knowledge regarding the specific animal) and/or behavioral (e.g. erratic or uncharacteristic movements) or vital signs parameters. The specific measurements of the animal (height, length, weight etc.) and relevant history may be loaded into the device and/or the system during a registration procedure. The unique identification data of the animal can also include: the pet animal's name, owner's names, personal details (address, phone number etc.), medical information concerning the pet and any other relevant data. The information may be included in the processing by processor 40 when the processor 40 analyzes data from the sensor elements 30.

A GPS device may be incorporated into collar 10. The GPS device could take the form, for example, of an integrated circuit or an RFID. Other location awareness technology may also be incorporated into the collar 10.

A receiving unit 68 attached to or incorporated into the collar 10 may be a smart phone, mobile (and/or hand-held) device, or any other communication/messaging device, or a specifically designed receiver or reader. The receiving unit 68 may be connected to the collar 10 in a wired and/or wireless manner as mentioned above. The receiving unit 68 may be detachable from the collar 10 for direct connection to a computer terminal 69 (FIG. 4), in order to enable faster or more secure downloading of stored (and in some cases processed) sensor data.

The collar 10 and/or system may gather analytical information including statistics, trend analysis, comparative analysis etc. regarding particular pets, particular breeds of pets or particular species of animals. The system may incorporate a social network for other animal owners for the purpose of sharing information.

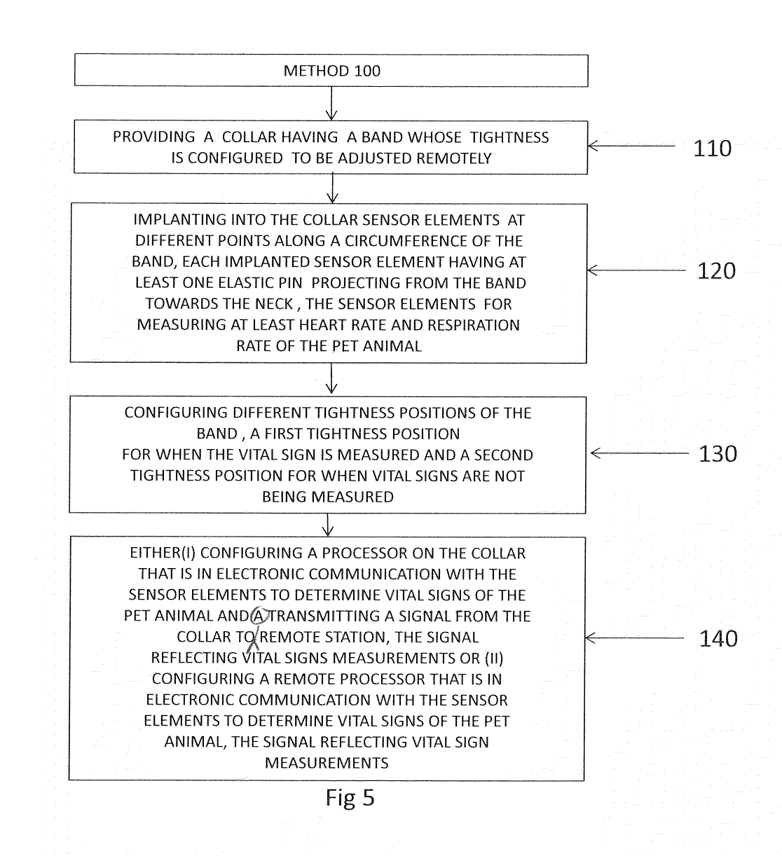

As shown in FIG. 5, the present invention may also be described as a method 100 of monitoring vital signs of a pet animal. A step 110 of method 100 may include providing a collar having a band whose tightness is configured to be adjusted. Preferably this adjustment can be made remotely. A further step 120 of method 100 may involve implanting into the collar 10 sensor elements 30 at different points along a circumference of the band. Each implanted sensor element 30 may have at least one elastic pin projecting from the band towards the neck of the pet animal and making contact with the neck of the animal. There could be two or three or four or more elastic pins for a particular sensor element instead of one. The sensor elements 30 may measure vital signs of the pet animal. In some embodiments, the vital signs include at least heart rate and respiration rate. In one version, one sensor element measures both of these. In a different version, one of the sensor elements may be for measuring heart rate, a second one of the sensor elements for measuring respiration rate, one may be for pulse, one may be for movement of the animal, one may be for measuring body temperature of the pet, and one may be for measuring the ambient temperature adjacent the pet or in an area near the pet. Other combinations are possible. The ambient temperature may be useful for getting a better understanding of the significance of the animal's body temperature or the animal's breathing rate or other vital signs.

Method 100 may have a step 130 of configuring different tightness positions of the band 20, preferably remotely. For example, one tightness position may be set, for example remotely, in preparation for one or more vital signs being measured. A second tightness position may be set, or it may be the default tightness position, for when vital signs are not being measured. A further step 140 of method 100 may involve either (i) configuring a processor on the collar that is in electronic communication with the sensor elements to interpret the sensor element data and determine vital signs of the pet animal and transmits a signal reflecting the vital sign determinations and measurements, from the collar to a remote station or (ii) configuring a remote processor that is in electronic communication with the sensor elements to determine vital signs of the pet animal, the signal reflecting vital sign measurements.

Method 100 may in some preferred embodiments have a further step of comprising reducing signal to noise ratio of the signal transmitted from the pet animal by including a layer of an elastic material on the collar to absorb noise from friction derived from movement of the pet animal's head. Animals tend to move their heads when walking and during any other movements such as when the animal is standing but moving. The signal to noise ratio may be significantly, if not dramatically reduced by absorbing the friction by means of the elastic layer on the band. This may allow the signal from the sensor array to be transmitted to the processor in a form that as a practical matter allows interpretation of the signal. The STN ratio reduction is particularly helpful for signals produced by the temperature sensor 30b. Since fur on the neck of the animal is an insulator against heat, measuring the body temperature of the pet animal is difficult. Since the signal derived from the temperature sensor is expected to be weak (due to the fur), it is that much more important for the noise to be lessened.

The method may also include, in some embodiments, a step of transmitting vital sign measurements to the pet owner, a veterinarian, a remote computer server or the authorities when the vital sign measurement exceeds a threshold level. Accordingly, processor 40 may be programmed to compare data received from the sensor elements to threshold levels of respiration rate, heart rate, temperature, movement, blood pressure, and/or other physiological data, such as noises made by a dog. Furthermore, the processor may have access to software in controller 49 that utilizes a function or a formula to relate combinations of the sensor element data. For example, if a dog moves in a certain way and utters a certain noise, that may trigger a particular alert or diagnosis. In addition, the programmer 40 may have access to its own data comparing the physiological data of a particular vital sign or combination of vital signs to the average vital sign data for pets of that species, that breed and that geographical location, taking into consideration the ambient temperature and the medical history of the pet. The controller/processor may transmit an alert to the pet owner, to a veterinarian or to the authorities.

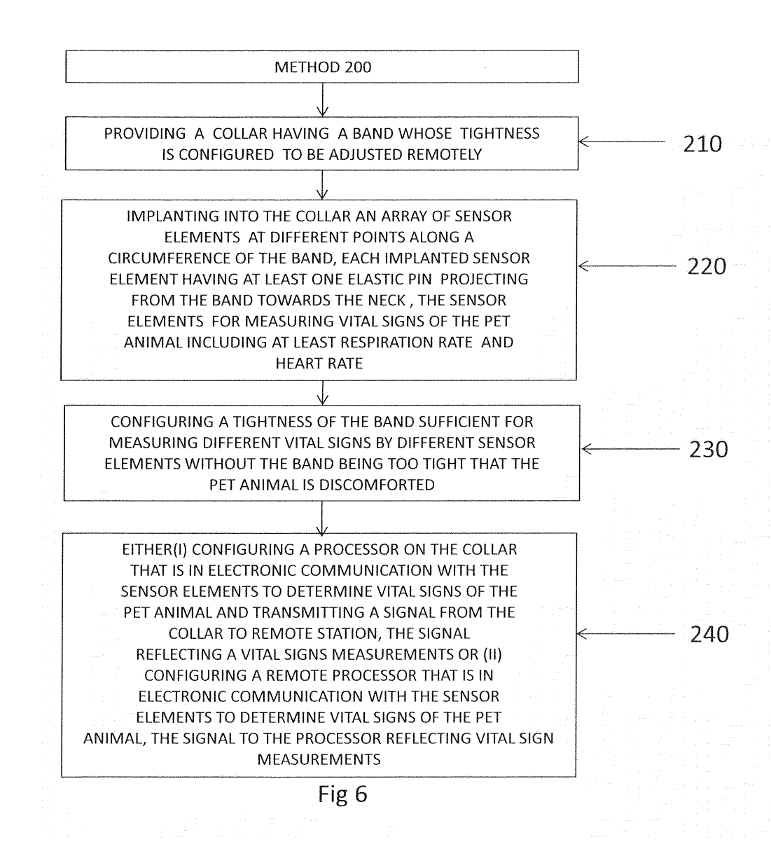

As shown in FIG. 6, in some preferred embodiments, the present invention is a method 200 of monitoring vital signs of a pet animal, comprising a step 210 of providing a collar having a band whose tightness is configured to be adjusted remotely. A further step 220 may involve implanting into the collar an array of sensor elements at different points along a circumference of the band, each implanted sensor element having at least one elastic pin projecting from the band towards the neck, the sensor elements for measuring a vital sign of the pet animal. One of the sensor elements may be for measuring heart rate, a second one of the sensor elements may be for measuring respiration rate, a third may be for measuring the pet's temperature, a further may be for measuring movement, a fifth may be for measuring ambient temperature. In other versions, the sensor array may include one (or two) sensor elements that measure at least heart rate and respiration rate. Other combinations are possible. The sensor array may also include a microphone for discerning and measuring noises of the pet and a speaker for communicating to the pet remotely.

Method 200 may also have a step 230 of configuring a tightness of the band sufficient for measuring one or more different vital signs by different sensor elements (or in some embodiments by the same sensor element) without the band being too tight that the pet animal is discomforted. Method 200 may include a step 240 of either (i) configuring a processor on the collar that is in electronic communication with the sensor elements to determine vital signs of the pet and transmitting a signal from the collar 10 (for example on a dog or cat) to a remote station, the signal reflecting vital sign measurements or (ii) configuring a remote processor that is in electronic communication with the sensor elements to determine vital signs of the pet animal, the signal to the processor reflecting vital sign measurements.

In one preferred embodiment of the collar 10 of the present invention, for monitoring vital signs of a pet animal, an adjustable tightness band 20 has a layer of an elastic material, the band for positioning on a neck of the pet animal. The collar may include at least one or at least two or at least three or at least four sensor elements at different points of the band, each sensor element having at least one elastic pin projecting from the band 20 towards the neck of the pet animal and having a power source, the array of sensor element for measuring at least two bioparameters (or in other preferred embodiments at least three or at least four) from temperature, heart rate, respiration rate, blood pressure, movement, each of the at least one or at least two or at least three or at least four sensor elements may be configurable remotely from the collar.

A processor 40 affixed to the collar 10 may be in electronic communication with each of the at least four sensor elements. The processor 40 may control a timing of an “ON” status of each sensor sufficient to trigger taking of a vital sign measurement. Memory storage 67 (FIG. 3) may be flash memory or other well known types of memory storage accessible by processor 40. The memory storage unit 67 may store data regarding the power requirements of each of the sensor elements in sensor array 30 as well as the lifespan of the battery 61 or other power source in collar 10. Alternatively, this data may be accessible by the processor 40 since processor 40 may be in communication with remote databases. As a result, the processor 40 may be configured to calculate the timing of the “ON” status of a sensor element (or of two or more or all the sensor elements) based on power requirements of the at least four sensors and a lifespan of the power source. In addition, processor 40 may receive sensor data from the sensor elements and communicate vital sign status of the pet animal to a remote location. The processor 40 may reach overall conclusions as to whether the pet has a particular medical condition by accessing databases and utilizing software containing diagnostic algorithms.

Particular features described in the context of one embodiment may be able to be incorporated into other embodiments for which that feature was not specifically mentioned. To take one example, while the release mechanism may have been described with respect to one particular embodiment, it may be applicable to any of the embodiments. Similarly, the two-way communication, the remote configurability of the tightness of the band 20, the pump and motor, the processor controller and their functionalities and other features may be applicable to all of the embodiments.

The following are non-limiting examples of vital sign and/or other physiological data for dogs acquired from sensor elements 30. In general, dog sounds recorded by the microphone 30c may be combined with information from other sensor elements 30 regarding dog postures and dog movements and this may be further combined with information from other sensor elements 30 such as temperature, respiration rate and pulse and other available data such as the time of day, the ambient temperature, the pet's normal behavior, the context etc. The processor 40 may reach conclusions about the presence of a high probability of medical conditions suffered by dogs or cats or other pet animals, such as hypothermia, hyperthermia, slow heart rate, normal or abnormal sinus arrhythmia, ear infections, torn ligaments, gastric dilatation, dyspnea, gastritis, pruritus and osteoarthritis. For example, hypothermia occurs when heat loss/output exceeds heat production. It can happen in cold weather, especially to small or sick animals, or under sedation or anesthesia. If low body temperature is recorded by the sensor elements 30 at a time when the ambient temperature is very cold, an alert may be sent. In another case, if a slower than normal heart rate is detected by sensor elements 30 in a pet animal the movements of the pet animal may be checked to determine if an alert needs to be sent. In general, the pulse rate may be compared to the respiration rate over time to see if the heart rate increases when the animal takes a breath. Regarding ear infections in a dog, if the sensor 30 input indicates movements consistent with an ear infections and the microphone sensor indicates sounds of pain when the ears are touched, an alert may be sent. Inflammation of the bones and joints is a common disease of older dogs. If the sensor input indicates decreased or change in activity relative to the time of day and sounds of pain, an alert may be transmitted.

While the invention has been described with respect to a limited number of embodiments, it will be appreciated that many variations, modifications and other applications of the invention may be made. Therefore, the claimed invention as recited in the claims that follow is not limited to the embodiments described herein.

What is claimed is:

1. A non-invasive collar for monitoring vital signs of a pet animal, wherein the pet animal is a dog or a cat, comprising:
a band having a layer of an elastic material, the band for positioning on a neck of the pet animal;
at least four sensor elements at different points of the band, each of the at least four sensor elements having at least one elastic pin projecting from the band towards the neck,
the at least four sensor elements comprising
(i) a first temperature sensor configured to measure ambient temperature and output a signal corresponding to ambient temperature,
(ii) a second temperature sensor configured to measure a skin temperature of the pet animal and output a signal representing vital sign data for skin temperature,
(iii) an acoustic sensor configured to measure at least one of heart rate and respiration rate and output at least one signal representing vital sign data for at least one of heart rate and respiration, and
(iv) at least one of a gyroscope, an accelerometer and a magnetometer, the at least one of the gyroscope, accelerometer and magnetometer configured to measure at least one of posture and movement and output a signal representing vital sign data for the at least one of posture and movement of the pet animal,
the collar, including the band and the at least four sensor elements, is configured to output the signals for the ambient temperature, skin temperature, at least one of heart rate and respiration and at least one of posture and movement, while being positioned on a single integrally connected body part of the pet animal.

2. The collar of claim 1, wherein the single integrally connected body part is the neck of the pet animal.

3. The collar of claim 1, wherein the collar in its entirety is on the neck of the pet animal.

4. The collar of claim 1, wherein the collar, including the band and the at least four sensor elements, is a single integrated unit.

5. The collar of claim 1, further comprising a processor configured to receive the signals representing the vital sign data and to execute software for processing the vital sign data using one or more combinations of the at least three vital sign bioparameters and the ambient temperature.

6. The collar of claim 1, the at least one of a gyroscope, an accelerometer and a magnetometer is configured to detect at least three of the following postures of a dog: lying down laterally, lying down sternally (head up/down), lying on back, sitting, standing on four legs, jumping, trotting, running, eating/drinking, urinating (male/female), defecating, limping hind leg, limping front leg, scratching hind leg, and stretching.

7. The collar of claim 1, wherein the at least one of a gyroscope, an accelerometer and a magnetometer is configured to detect at least four of the following postures of a dog: lying down laterally, lying down sternally (head up/down), lying on back, sitting, standing on four legs, jumping, trotting, running, eating/drinking, urinating (male/female), defecating, limping hind leg, limping front leg, scratching hind leg, and stretching.

8. The collar of claim 1, wherein the at least one of a gyroscope, an accelerometer and a magnetometer is configured to detect at least five of the following postures of a dog: lying down laterally, lying down sternally (head up/down), lying on back, sitting, standing on four legs, jumping, trotting, running, eating/drinking, urinating (male/female), defecating, limping hind leg, limping front leg, scratching hind leg, and stretching.

9. The collar of claim 1, wherein the at least one of a gyroscope, an accelerometer and a magnetometer is configured to detect at least seven of the following postures of a dog: lying down laterally, lying down sternally (head up/down), lying on back, sitting, standing on four legs, jumping, trotting, running, eating/drinking, urinating (male/female), defecating, limping hind leg, limping front leg, scratching hind leg, and stretching.

10. The collar of claim 1, wherein the at least one of a gyroscope, an accelerometer and a magnetometer is configured to detect at least nine of the following postures of a dog: lying down laterally, lying down sternally (head up/down), lying on back, sitting, standing on four legs, jumping, trotting, running, eating/drinking, urinating (male/female), defecating, limping hind leg, limping front leg, scratching hind leg, and stretching.

11. The collar of claim 1, wherein the at least one of a gyroscope, an accelerometer and a magnetometer is configured to detect at least twelve of the following postures of a dog: lying down laterally, lying down sternally (head up/down), lying on back, sitting, standing on four legs, jumping, trotting, running, eating/drinking, urinating (male/female), defecating, limping hind leg, limping front leg, scratching hind leg, and stretching.

12. The collar of claim 1, wherein the at least one of a gyroscope, an accelerometer and a magnetometer is configured to detect at least the following postures of a dog: lying down laterally, lying down sternally (head up/down), lying on back, sitting, standing on four legs, jumping, trotting, running, eating/drinking, urinating (male/female), defecating, limping hind leg, limping front leg, scratching hind leg, and stretching.

13. The collar of claim 1, further comprising a two-way communication device for communication to and remote monitoring of the pet animal's health by a remotely stationed veterinarian.

14. The collar of claim 1, wherein the acoustic sensor is configured to measure both heart rate and respiration rate.

15. The collar of claim 1, wherein the at least one of the gyroscope, accelerometer and magnetometer is configured to measure both posture and movement.

16. The collar of claim 1, further comprising a microphone for listening to special noises of the pet animal and a speaker for communicating sounds to the pet animal remotely.

17. The collar of claim 1, further comprising a memory storage for storing health information history of the pet animal, the memory storage accessible by the processor.

18. The collar of claim 1, further comprising a processor for executing algorithms to interpret an interdependence of the vital sign data and ambient temperature from the at least four sensor elements and arrive at a tentative diagnosis.

19. The collar of claim 1, wherein the pet animal is a dog.

20. The collar of claim 1, wherein the pet animal is a cat.
